(19) United States
(12) Patent Application Publication
(10) Pub. No.: US 2020/0330561 A1
(43) Pub. Date: Oct. 22, 2020

(54) COMPOSITION FOR TREATMENT OF FACIAL NERVE PALSY

(71) Applicant: Nobelpharma Co., Ltd., Tokyo (JP)

(72) Inventors: Seiji KAKEHATA, Yamagata (JP); Tsukasa ITO, Yamagata (JP); Takatoshi FURUKAWA, Yamagata (JP); Motoyasu SUGIYAMA, Yamagata (JP); Takanari GOTO, Yamagata (JP)

(21) Appl. No.: 16/433,797

(22) Filed: Jun. 6, 2019

(30) Foreign Application Priority Data
Apr. 16, 2019 (JP) 2019-077660

(51) Int. Cl. A61K 38/30; A61K 47/42; A61K 47/69; A61P 25/00

(52) U.S. Cl. CPC A61K 38/30; A61P 25/00; A61K 47/6903; A61K 47/42

(57) ABSTRACT

Object:

To provide a composition for treatment of facial nerve palsy having less invasiveness and sufficient therapeutic effect.

Resolution means:

A composition and a kit for treatment of facial nerve palsy in which a substance having a nerve regeneration effect is carried on a carrier made of a bioabsorbable polymer. In particular, a composition for treatment of facial nerve palsy in which an insulin-like growth factor 1 (IGF-1) is carried on a gelatin sponge and a kit capable of preparing the composition.

### TECHNICAL FIELD

The present invention relates to a composition for treatment of facial nerve palsy in which a substance having a nerve regeneration effect is carried on a carrier made of a bioabsorbable polymer. The present invention also relates to a kit for treatment of facial nerve palsy including a substance having a nerve regeneration effect and a carrier made of a bioabsorbable polymer.

### BACKGROUND ART

Peripheral facial nerve palsy considerably affects appearance, and therefore patients with peripheral facial nerve palsy may be socially isolated. Thus, the QOL of the patients may be often reduced. Accordingly, peripheral facial nerve palsy can be one of diseases for which therapy is highly required.

Approximately 80 to 90% of facial nerve palsy is Bell's palsy or Hunt syndrome. Facial nerve palsy is considered to arise as follows. In the facial nerve canal located inside the temporal bone, the facial nerve is inflamed by influence of virus, and strangulated, resulting in an ischemic condition. In recent years, administration of high-dose steroid with an antiviral agent, which uses an anti-inflammatory effect of steroid, has been performed for facial nerve palsy, and a certain effect is obtained (Non-Patent Document 1). However, this method has a problem in which the effect is not seen in some patients. Further, this method has problems in which side effects on the body are severe and a steroid cannot be used in the case where complication arises.

For a severe palsy case, surgical decompression of the facial nerve, which is a microscopic surgery under general anesthesia, may be added as a salvage therapy. However, this method is highly invasive to patients, and is not necessarily a therapy in which sufficient therapeutic effects are expected. Currently, this method is hardly performed in Europe and the U.S. because this method has higher invasion degree and less benefit to be obtained (Non-Patent Document 2).

On the other hand, various methods for treatment of facial nerve palsy have been considered and reported. For example, it is reported that in an experiment using a rat facial nerve cut model, regeneration of the facial nerve is confirmed by continuous administration of insulin-like growth factor 1 (hereinafter referred to as IGF-1) to an affected area (Non-Patent Document 3).

Recently, it is also reported that after postauricular incision, a basic fibroblast growth factor (hereinafter referred to as bFGF) carried on gelatin hydrogel is placed at an affected area, achieving a therapeutic effect for human Bell's palsy (Non-Patent Document 4).

### Non-Patent Documents

Non-Patent Document 1: Furukawa T., et al., “Benefits of High-dose Steroid+Hespander+Mannitol Administration in the Treatment of Bell's Palsy,” Otology&Neurotology, Volume 38, Issue 2(2017), p. 272
Non-Patent Document 2: Smouha E et al., “Surgical treatment of Bell's palsy: current attitudes,” J LARYNGOSCOPE, VOLUME 121, NUMBER 9(2011), p. 1965
Non-Patent Document 3: Panayotis K. Thanos et al., “Insulin-like growth factor-I promotes nerve regeneration through a nerve graft in an experimental moel of facial paralysis”, Restorative Neurology and Neuroscience 15(1999), p. 57
Non-Patent Document 4: Naohito Hato et al., “Facial Nerve Decompression Surgery Using bFGF-Impregnated Biodegradable Gelatin Hydrogel in Patients with Bell Palsy.”, Otolaryngology-Head and Neck Surgery, 146 (4), (2012), p. 641

### SUMMARY OF THE INVENTION

### Problems to be Solved by the Invention

As described above, various therapies for facial nerve palsy have been proposed. However, a method for administering a high-dose steroid with an antiviral agent has a problem in which the effect is not seen in some patients. In addition, this method has problems in which side effects on the body are severe and a steroid cannot be used in the case where complication arises. Surgical decompression of the facial nerve has a problem of high invasiveness.

A method in which IGF-1 is continuously administered to the affected area, as described in Non-Patent Document 3, has a problem of high invasiveness. In addition, this method has a problem of lack of general-purpose because a pump needs to be used for continuous administration. Further, the model used is a model in which the nerve is cut outside the temporal bone. Therefore, an actual clinical case of facial nerve palsy is not applied. Conditions for treatment of facial nerve palsy with IGF-1 are not disclosed or suggested.

In the method described in Non-Patent Document 4, postauricular incision is required. In consideration of a method widely performed in a clinical field, use of a less-invasive method is desired.

In view of the above circumstances, an object of the present invention is to provide a composition and a kit for treatment of facial nerve palsy having less invasiveness to a patient and higher therapeutic effect.

### Means for Solving the Problems

The present inventors have intensively studied, and as a result, found that when a composition in which a substance having a nerve regeneration effect such as IGF-1 is carried on a carrier made of a bioabsorbable polymer formed into a shape which is suitable for placement in the tympanic cavity is used, the aforementioned problems can be solved. Thus, the present invention has been accomplished.

Specifically, the present invention is a composition for treatment of facial nerve palsy which is inserted through the external auditory canal and placed at an opening area of the facial nerve canal positioned so as to deliver the composition from the tympanic cavity to the facial nerve. In the composition, a substance having a nerve regeneration effect is carried on a carrier made of a bioabsorbable polymer.

The facial nerve reaches the face from the brain stem through the facial nerve canal in the temporal bone. The facial nerve is adjacent to the tympanic cavity via a thin area of the temporal bone. Therefore, when a small opening area is provided from the tympanic cavity to the temporal bone, a route from the tympanic cavity to the facial nerve can be secured. The present invention is the composition for treatment of facial nerve palsy which is used so as to be placed at this opening area. The composition is characterized that a substance having a nerve regeneration effect is carried on a carrier made of a bioabsorbable polymer. When the substance having a nerve regeneration effect is carried on the carrier made of a bioabsorbable polymer, the substance having a nerve regeneration effect can be continuously delivered to the facial nerve for a fixed period. According to the present invention, less invasiveness and higher therapeutic effect for facial nerve palsy can be achieved.

Further, the present invention is a kit for treatment of facial nerve palsy including the substance having a nerve regeneration effect and the bioabsorbable polymer.

### Effects of the Invention

According to the present invention, a composition and a kit for treatment having less invasiveness and higher therapeutic effect for facial nerve palsy can be obtained.

### BRIEF DESCRIPTION OF DRAWINGS

FIG. 1 is a drawing illustrating the distance between upper and lower eyelids measured in measurement of degree of eye closure.

FIG. 2 is a view illustrating a change in degree of eye closure over time in an experiment system in which tight eye closure is assumed.

FIG. 3 is a view illustrating a change in degree of eye closure over time in an experiment system in which light eye closure is assumed.

FIG. 4 is a view illustrating a result of measurement of ENoG value in an IGF-1 administration model and a control model.

### DESCRIPTION OF EMBODIMENTS

In a composition for treatment of facial nerve palsy of an embodiment of the present invention, a substance having a nerve regeneration effect (hereinafter referred to as a nerve regeneration substance) is carried on a carrier made of a bioabsorbable polymer which has a shape suitable for placement in the tympanic cavity.

In an embodiment of the present invention, the nerve regeneration substance which is an active ingredient and is carried on the bioabsorbable polymer is not particularly limited as long as it may be a substance providing a nerve regeneration effect. Specifically, IGF-1, bFGF, a hepatocyte growth factor (HGF), a glial cell line-derived neurotrophic factor (GDNF), or the like can be used. Of these, IGF-1 can be preferably used.

The bioabsorbable polymer is not particularly limited as long as it has bioabsorbable property and is allowed to prepare a carrier capable of carrying the nerve regeneration substance (e.g., porous carrier). Examples thereof include gelatin and chitosan. Of these, gelatin can be preferably used. A carrier made of a bioabsorbable molecule is a carrier made of the bioabsorbable polymer. A gelatin sponge can be preferably used.

Hereinafter, the composition for treatment of facial nerve palsy of an embodiment of the present invention will be specifically described using an IGF-1-carrying gelatin sponge as an example.

Method for preparing composition for treatment of facial nerve palsy A gelatin sponge is obtained by processing gelatin extracted from pig, cow, or the like into a porous structure. As a material for preparing a gelatin sponge, the gelatin obtained by a known method, in which collagen derived from an animal such as pig or cow is hydrolyzed with an acid or an alkali or decomposed by heat to obtain a protein and the protein is purified, can be used. In general, such gelatin is commercially available. The type of gelatin used in an embodiment of the present invention is not particularly limited. It is desirable that “gelatin” in the Japanese pharmacopoeia or “purified gelatin” in the Japanese pharmacopoeia be used.

A gelatin sponge can be prepared by a known method (e.g., the method described in “WO 2009/157558”). Specifically, a gelatin sponge can be obtained by steps of (I) dissolving gelatin in heated water and filtering the solution through a filter having a pore diameter of 0.2 μm while the temperature is maintained at 45° C. or higher, (II) vigorously stirring the obtained gelatin aqueous solution by a homogenizer or the like, to generate bubbles, (III) immediately lyophilizing the bubbled gelatin aqueous solution by a known method (e.g., under a condition where the lyophilized solution is disposed under a reduced pressure of 0.1 Torr), and (IV) cutting the resultant lyophilizate into a sheet having desired thickness (e.g., 1 cm). In addition to the aforementioned steps, a step of (V) heating the obtained sheet to thermally cross-link the gelatin (e.g., heating the sheet at 150° C. for about 6 hours) may be added. Through this step, the strength of the resulting gelatin sponge can be enhanced, and thus, a composition having improved stability can be obtained. The gelatin concentration in the gelatin aqueous solution is appropriately adjusted so as to have desired physical properties. Specifically, the gelatin concentration is adjusted so that the moisture absorption in a water absorption test (a value obtained by dividing the mass after impregnation with water by the mass before the impregnation) is about 40 to 50 times. The gelatin concentration can be usually 5.5 to 6.5%.

IGF-1 can be carried on the gelatin sponge by adding dropwise an aqueous solution of IGF-1 to the gelatin sponge cut into a desired shape (e.g., a cylinder having a diameter of about 1.5 cm and a thickness of about 1 cm) or impregnating the gelatin sponge with the aqueous solution of IGF-1. The shape and size of the gelatin sponge are adjusted as appropriate depending on the condition of a placement area. Specifically, the gelatin sponge is cut into a suitable size (e.g., a size sufficient to cover the opening area provided in the tympanic cavity) by an operator depending on the volume of the middle ear cavity or the condition of the round window niche in each case and a required amount of IGF-1 is carried on the gelatin sponge.

The amount of IGF-1 to be carried is appropriately adjusted depending on the condition and the like of a patient. In general, the gelatin sponge cut into the shape of the affected area may be impregnated with a saline solution of IGF-1 in a dose (10 mg/mL) adjusted per administration, and used. Depending on the condition of the patient, administration to the affected area may be performed a plurality of times.

Method for using composition for treatment of facial nerve palsy The composition for treatment of facial nerve palsy of an embodiment of the present invention may be used so as to be directly placed at an opening area of the facial nerve canal provided in the tympanic cavity. Specifically, a case of an IGF-1-carrying gelatin sponge will be described by way of example. Under local anesthesia, the external auditory canal is incised in a semicircular shape and the eardrum is released from the skin. The lateral wall on the side of the facial nerve canal in the tympanic cavity is incised, and the temporal bone is punctured to open the facial nerve canal. The IGF-1-carrying gelatin sponge which is cut into an appropriate size is placed at the opening area by using a special instrument. From the placed IGF-1-carrying gelatin sponge, IGF-1 is gradually infiltrated into the affected area. Thus, facial nerve palsy can be effectively cured.

### OTHER EMBODIMENTS

In the composition for treatment of facial nerve palsy of an embodiment of the present invention, IGF-1 or a nerve regeneration substance such as bFGF, a hepatocyte growth factor (HGF) or a glial cell line-derived neurotrophic factor (GDNF) may be carried on a carrier made of a bioabsorbable polymer by the same method as in a case of carrying IGF-1.

As a carrier made of a polymer having bioabsorbable property, a gelatin sponge or a carrier prepared from chitosan may be also used. Herein, the carrier prepared from chitosan can be obtained by lyophilizing a chitosan solution or gel which is obtained by a known method, by a known method. When the chitosan solution is lyophilized, it is desirable that the solution be bubbled by a means such as stirring and then lyophilized in the same manner as in a case of a gelatin sponge.

Kit for Treatment of Facial Nerve Palsy

A kit for treatment of facial nerve palsy of an embodiment of the present invention can be produced by disposing the aforementioned nerve regeneration substance and the carrier made of a bioabsorbable polymer in a known container. For example, IGF-1 as a nerve regeneration substance and a gelatin sponge as a carrier made of a bioabsorbable polymer are disposed in a known container. When IGF-1 is a lyophilizate, the kit may further include a saline for dissolution, water for injection, syringe, an instrument for cutting a gelatin sponge into an appropriate size, a tray for carrying IGF-1 on a gelatin sponge, or the like. In this case, the kit for treatment of facial nerve palsy of an embodiment of the present invention is used so that a substance having a nerve regeneration effect contained in the kit is dissolved in a saline solution, a gelatin sponge is impregnated with the solution to prepare the composition for treatment of facial nerve palsy according to an embodiment of the present invention, and the composition is placed at an opening area in the tympanic cavity by the same procedure as described above.

### EXAMPLES

(1) Model Animal Creation

A Hartley guinea pig (4-week-old, male) was prepared, a postauricular region was incised, and the otic capsule and the main trunk of the facial nerve were identified. After then, ostectomy was partially performed from the posterior otic capsule to the stylomastoid foramen. The bone at the stylomastoid foramen was removed using a cup-shaped forceps or the like, to expose a descending area of the facial nerve. In the temporal bone, the exposed facial nerve was clamped by a micro forceps (BM563R Castroviejo), to create a guinea pig of intratemporal bone facial nerve strangulation model.

(2) IGF-1-Carrying Gelatin Hydrogel Preparation

In 40 μL of saline solution, 0.4 mg of IGF-1 (available from OrphanPacific, Inc.) was dissolved to prepare an IGF-1 solution. With the obtained IGF-1 solution, 4 mg of dry gelatin hydrogel (trade name: MedGel (PI5) available from MedGEL CO., LTD) was impregnated to prepare an IGF-1-carrying gelatin hydrogel.

(3) IGF-1-Carrying Gelatin Hydrogel Placement at the Affected Area

The prepared IGF-1-carrying gelatin hydrogel was locally placed in the tympanic cavity so as to cover the facial nerve, and a wound was closed (hereinafter referred to as IGF-1 administration model).

As a control, a treatment in the same manner as described above was performed using a gelatin hydrogel impregnated with a saline solution instead of IGF-1, to create an animal in which a wound was closed (hereinafter referred to as control model).

(4) Confirmation of Therapeutic Effect

1) Measurement of Degree of Eye Closure

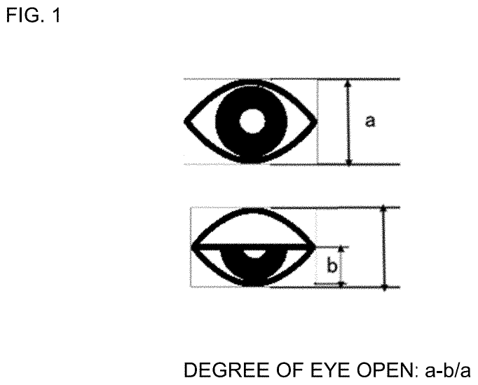

Air (wind speed: approximately 0.28 m/s (3 cm), 0.19 m/s (6 cm)) was blown around the operated eye from distances of 3 cm and 6 cm to induce eye closure, and the situation was recorded on video at 60 fps. A longitudinal length a of the eye before blowing air and a distance b between a lower part of the eye and the end of the eyelid during blowing air were measured. The degree of eye closure, defined as (a-b)/a, was determined (FIG. 1). Herein, an experiment where air was blown from a distance of 3 cm was assumed to be tight eye closure, and an experiment where air was blown from a distance of 6 cm was assumed to be light eye closure. The measurement was performed once per week from four weeks to eight weeks after the operation. For the control model and the IGF-1 administration model, a change in degree of eye closure over time was examined. The experiment was performed for six animals per group.

A case where in the eighth week after the operation, the degree of eye closure is 100% is defined as complete recovery, and a case where in the eighth week after the operation, the degree of eye closure is less than 100% is defined as incomplete recovery. The numbers of completely recovered animals in the control model and the IGF-1 administration model were compared.

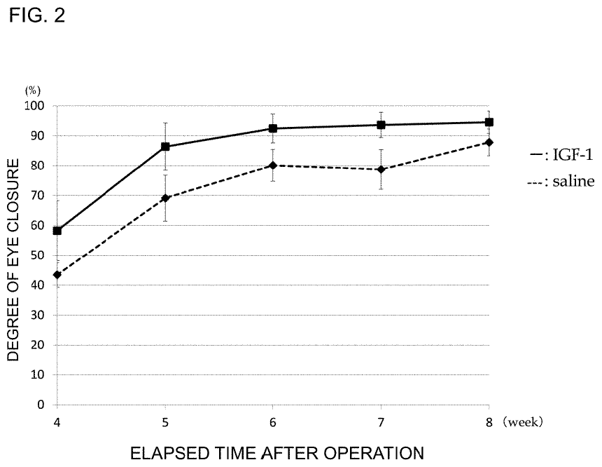

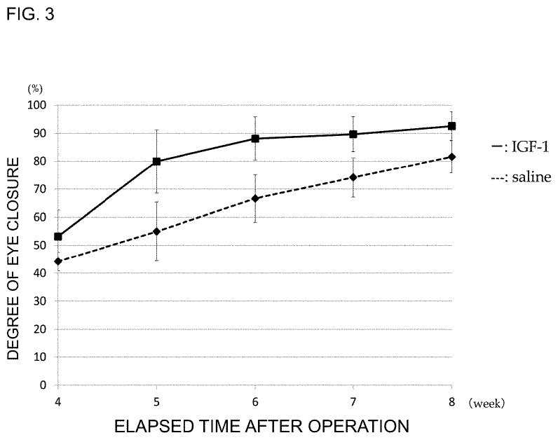

FIGS. 2 and 3 illustrate changes in degree of eye closure over time in the experiments where tight eye closure and light eye closure, respectively, are assumed.

As illustrated in FIGS. 2 and 3, the IGF-1 administration model exhibited a tendency to recover the degree of eye closure in the experiments of both tight eye closure and light eye closure as compared with the control model. As shown in Table 1, animals of the control model which were completely recovered in the eighth week after the operation were not confirmed in the experiments of both tight eye closure and light eye closure. Among six animals of the IGF-1 administration model, four animals were confirmed to be completely recovered.

TABLE 1
Number of completely recovered animals which
are confirmed from degree of eye closure

| | | Complete | Incomplete |
|---|---|---|---|
| | | recovery | recovery |
| | | (degree of eye | (degree of eye |
| | Model | closure = 100%) | closure < 100%) |
| Tight eye | Control model | 0 | 6 |
| closure group | IGF-1 adminis- | 4 | 2 |
| | tration model | | |
| Light eye | Control model | 0 | 6 |
| closure group | IGF-1 adminis- | 4 | 2 |
| | tration model | | |

2) Electrophysiological Evaluation

An electrode was attached to the skin on the muscles of facial expression on an operation side and an unaffected side, a compound muscle action potential of the nose was measured by an electromyogram machine (trade name: Power Lab 26T, available from Bio Research Center Co., Ltd.), and an ENoG value (%) was calculated. The ENoG value was calculated in eight weeks after placement of the IGF-1-carrying gelatin hydrogel.

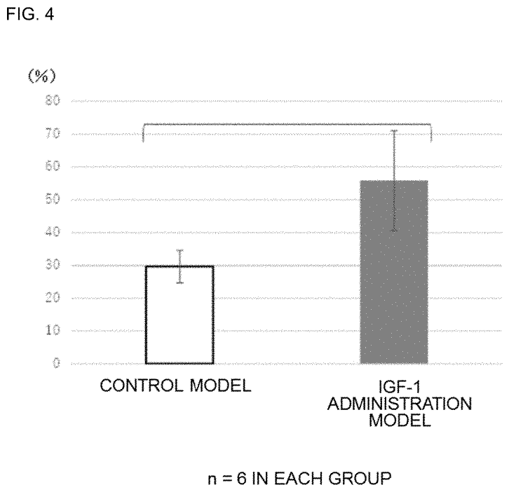

The measurement results of the ENoG value is illustrated in FIG. 4. As illustrated in FIG. 4, the ENoG value for the IGF-1 administration model is higher than that for the control model.

The aforementioned results show that when an IGF-1-carrying carrier is placed in the tympanic cavity where the facial nerve is exposed, facial nerve palsy is recovered.

### INDUSTRIAL APPLICABILITY

By using the composition and kit for treatment of facial nerve palsy of the present invention, a pharmaceutical having low invasiveness and capable of effectively treating facial nerve palsy can be provided.

1. A composition for treatment of facial nerve palsy which is inserted through the external auditory canal and placed at an opening area of the facial nerve canal positioned so as to deliver the composition from the tympanic cavity to the facial nerve, wherein a substance having a nerve regeneration effect is carried on a carrier made of a bioabsorbable polymer.

2. The composition for treatment of facial nerve palsy according to claim 1, wherein the carrier made of a bioabsorbable polymer is a gelatin sponge.

3. The composition for treatment of facial nerve palsy according to claim 1, wherein the substance having a nerve regeneration effect is an insulin-like growth factor 1 (IGF-1).

4. The composition for treatment of facial nerve palsy according to claim 2, wherein the substance having a nerve regeneration effect is an insulin-like growth factor 1 (IGF-1).

5. A kit for treatment of facial nerve palsy comprising a substance having a nerve regeneration effect and a carrier made of a bioabsorbable polymer.

6. The kit for treatment of facial nerve palsy according to claim 5, wherein the substance having a nerve regeneration effect is an insulin-like growth factor 1 (IGF-1) and the carrier made of a bioabsorbable polymer is a gelatin sponge.
